(19) United States
(12) Patent Application Publication
(10) Pub. No.: US 2007/0065896 A1
(43) Pub. Date: Mar. 22, 2007

(54) Methods and kits for quantifying metabolites or analytes

(76) Inventors: Graham Herbert Coombs, Glasgow (GB); Jeremy Charles Mottram, Glasgow (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/558,240

(22) PCT Filed: May 21, 2004
(86) PCT No.: PCT/GB04/02227

§ 371 Date: Sep. 22, 2006

(30) Foreign Application Priority Data
May 22, 2003 (GB) 0311804.9

(51) Int. Cl. C12Q 1/37; C12Q 1/26

(52) U.S. Cl. 435/23; 435/25

(57) ABSTRACT

Methods and kits for quantifying the amount of a metabolite, such as homocysteine, methionine or cysteine, in a sample are disclosed. This involves contacting the sample with a first enzyme capable of degrading a metabolite into a plurality of reaction products and regenerating the metabolite using a second enzyme which is capable of converting at least one of said reaction product(s), together with an additional substrate or substrates, into said metabolite. This process optionally generates an additional by-product or by-products. After repeating this process, the metabolite is quantified by detecting a level of a reaction product formed by enzyme degradation of said metabolite by said first enzyme, wherein the reaction product is different from the reaction product used in regenerating said metabolite, or detecting a level of said optional by-product produced by said second enzyme or detecting a level of an additional substrate. The method may also involve the initial conversion of an analyte in a sample to a metabolite by contacting the analyte with a reducing agent. The present method further provides a method of quantifying the amount of an analyte in sample by reacting the analyte to produce a metabolite in a cyclic reaction.

### FIELD OF THE INVENTION

The present invention provides a method of quantifying the amount of a metabolite in a sample by amplifying an amount of detectable product which comprises recycling a degradation product of the hydrolysis reaction of metabolite. The present method further provides a method of quantifying the amount of an analyte in sample by reacting the analyte to produce a metabolite in a cyclic reaction. In preferred applications said metabolite is homocysteine, methionine or cysteine.

### BACKGROUND OF THE INVENTION

An elevated level of homocysteine in the blood appears to be an important indicator for many human disease states. Homocysteine is predictive of vascular disease and stroke, Ueland, P. M. (1992) and Kluijtmans L. A. J. et al (1996); is correlated with forms of diabetes and alcoholism, Cravo, M. L. et al (1996); is used to monitor liver and kidney damage, Bostom, A. G. et al (1996) and neural tube defects, Steegers-Theunissen, R. P. N. (1992) and is associated with certain inborn errors of metabolism, Mudd, S. H., (1989) and neurodegenerative conditions such as Alzheimer's disease. More details are provided at http://www.homocysteine.net/.

Homocysteine levels in blood are conventionally determined using high performance liquid chromatography (HPLC) methods, see for example Poele-Pothoff M. T. et al, (1995), and fluorescence polarisation immunoassay [FPIA] using Abbott IMX (Shipchandler and Moore, 1995) or enzyme immunoassay [EIA] using Axis kit (Frantzen et al., 1998). However, HPLC methods employ expensive and elaborate machinery, are generally sophisticated and are considered impractical for many routine analyses. Patent publication WO 93/15220 (Cockbain) describes a method for assaying homocysteine in blood using a homocysteine converting enzyme, S-adenosyl homocysteine hydrolase (SAH-hydrolase). SAH-hydrolase catalyses the conversion of homocysteine with a co-substrate adenosine to S-adenosyl-homocysteine. It is then possible, by determining the amount of adenosine consumed, to make a correlation with the amount of homocysteine consumed. The amount of homocysteine in a sample is then determined from differences in adenosine concentration. However, such an assay involves determining a decrease in the concentration of adenosine, which may not be satisfactory.

U.S. Pat. No. 5,438,017 describes a gas chromatography/mass spectrometry method for analysis of sulphydryl amino acids in a sample of body fluid. The assay relies on the use of a labelled reference sulphydryl amino acid, similar to that described in U.S. Pat. No. 4,940,658, but has additional treatment and/or purification steps prior to analysing the sample by gas chromatography/mass spectrometry.

It will be appreciated that similar to HPLC methods, the assays described above which employ gas chromatography/mass spectrometry are generally sophisticated, use expensive and elaborate machinery and are considered impractical for many routine analyses.

A simpler, direct enzymatic assay has previously been described by the present inventors in WO 98/07872. For example, the enzyme homocysteine desulphurase has been used to catalyse the degradation of homocysteine to the reaction products α-ketobutyrate, hydrogen sulphide and ammonia. The reaction products can then be detected by standard methods and the level of homocysteine in a sample quantified in relation to the level of any of the reaction products. Nevertheless, such a direct assay may produce relatively low levels of reaction product(s), which may prove difficult to detect and quantify in some instances.

There is a continuing need in the art to develop assays for the metabolites, homocysteine, methionine or cysteine which obviates and/or mitigates at least one of the abovementioned disadvantages and/or which facilitates a simpler and more accurate assay for the detection of the abovementioned metabolite concentrations.

### SUMMARY OF THE INVENTION

Accordingly, in a first aspect, the present invention provides a method for assaying a metabolite in a sample comprising the steps of:

(a) contacting the sample with a first enzyme capable of degrading a metabolite into a plurality of reaction products; and

(b) regenerating said metabolite using a second enzyme which is capable of converting at least one of said reaction product(s) obtained in a), together with an additional substrate or substrates, into said metabolite, and optionally generating an additional by-product or by-products;

(c) repeating steps a) and b) for a period of time; and

(d) thereafter detecting a level of a reaction product formed by enzyme degradation of said metabolite by said first enzyme, wherein the reaction product is different from the reaction product used in regenerating said metabolite, or detecting a level of said optional by-product produced by said second enzyme, or detecting a level of an additional substrate.

Conveniently, steps a) and b) of the method of the present invention may be essentially carried out simultaneously. That is, the first and second enzymes together with the additional substrate may be contacted with the sample at substantially the same time.

The metabolite to be assayed is typically homocysteine, methionine or cysteine. However, for the sake of simplicity and brevity, reference hereinafter will generally be made to homocysteine, but this should not be construed as limiting.

It should be understood that it is also possible to assay one of said additional substrates, which may be present in the sample. Examples of additional substrates include O-acetyl-L-homoserine or O-succinyl-L-homoserine wherein the metabolite is homocysteine or methionine; and O-acetyl-L-serine or O-succinyl-L-serine, wherein the metabolite is cysteine. This will be described in more detail hereinafter.

It will be understood that “cyclical” methods of the present invention, for the assay of a metabolite by enzymatic means, generally requires at least two enzymes which recycle at least one reaction product produced by the degradation of a metabolite by a first enzyme; and a second enzyme may then recycle at least one of said reaction products in the presence of an additional substrate(s) in order to regenerate the starting metabolite. Both of said first and second enzymes may be provided in a sufficient concentration such that the metabolite is irreversibly converted into said reaction products. Optionally, a by-product may be produced on the reaction of the second enzyme with the additional substrate(s). Thus, an amount of at least one of the reaction products, and/or said optional by-product may therefore be amplified by the recycling process.

After a period of time, at least one of said reaction products, which is different to the reaction product used to regenerate said metabolite, or said optional by-product, defined according to the present invention, may be detected and the amount of metabolite in the sample quantified as described herein. Advantageously, said reaction product(s), which is/are different to the reaction product used to regenerate said metabolite, or said optional by-product is/are amplified and therefore due to their amplified concentration in said sample can be more easily and accurately detected and quantified as compared to reaction products of a “non-cycling” method for the assay of a metabolite. Typically, the amount of metabolite present in the sample is quantified by reference to a “standard curve” generated using a number of standards comprising known concentrations of metabolites, as well known to those skilled in the art.

Conveniently, the amount of amplification of said metabolite and said reaction product(s) in such an assay is 2- to 1,000-fold, such as 50- to 750-, 100- to 500-, and 10- to 100-fold.

The term reaction product(s) according to the present invention is/are understood to mean any product formed through the degradation of the metabolites, homocysteine, methionine, or cysteine by a suitable (first) enzyme. For example, when the (first) enzyme suitable for degrading such substrates is homocysteine desulphurase (also sometimes known as methionine-γ-lyase or methioninase) the reaction products of the following metabolites are:

Homocysteine:—α-ketobutyrate, ammonia (NH₃) and hydrogen sulphide (H₂S).

Methionine:—α-ketobutyrate, ammonia and methanethiol.

Cysteine:—ammonia, hydrogen sulphide and pyruvate.

Preferably, the (first) enzyme capable of degrading homocysteine in a sample is homocysteine desulphurase, for example recombinant homocysteine desulphurase as described in WO 98/07872 or any enzymatically active fragment or derivative thereof.

An enzymatically active fragment or derivative thereof according to the present invention is understood to mean any truncated product, whether man-made (recombinant) or natural which retains enzymatic activity, i.e. homocysteine desulphurase activity. The skilled person will understand that homocysteine desulphurase activity may be determined and tested using known and standard means. Furthermore, it should be understood that the enzyme homocysteine desulphurase is also known in the art as methionine-γ-lyase and therefore use of all versions of this enzyme are intended to be encompassed herein. For simplicity, reference will generally be made to homocysteine desulphurase. However, this should not be construed as limiting.

It should be understood that homocysteine desulphurase according to the present invention may be used to assay and degrade other substrates in a sample, including methionine and cysteine. Therefore, it should be understood that the cyclical method of the present invention may also be used to assay methionine and/or cysteine, wherein the first enzyme is homocysteine desulphurase as defined herein.

“Homocysteine desulphurase” as used herein refers to an enzyme that is capable of catalysing the degradation of homocysteine to release α-ketobutyrate, hydrogen sulphide, and ammonia.
HS—CH₂—CH₂—CH(NH₂)COOH+H₂O→CH₃—CH₂—C(O)COOH+H₂S+NH₃

Preferably, the (second) enzyme capable of resynthesising the metabolites, homocysteine, methionine or cysteine from any of said reaction product(s) may be any enzyme which utilises any of said reaction product(s) in the presence of a further substrate in the synthesis of said metabolite(s).

For example, for the reaction product hydrogen sulphide any enzyme that utilises hydrogen sulphide in the synthesis of homocysteine or cysteine may be used in the method of the present invention. Alternatively, for the reaction product methanethiol, any enzyme that utilises methanethiol in the synthesis of methionine may be used in the method of the present invention.

More preferably, the (second) enzyme capable of resynthesising the reaction product, hydrogen sulphide to synthesise homocysteine is O-acetyl-L-homoserine thiol lyase and said additional substrate is O-acetyl-L-homoserine or O-succinyl-L-homoserine. More preferably, the second enzyme capable of resynthesising the reaction product methanethiol to synthesise methionine is O-acetyl-L-homoserine thiol lyase and said additional substrate is O-acetyl-L-homoserine or O-succinyl-L-homoserine. It should be understood that O-acetyl-L-homoserine thiol lyase is also known as O-acetyl-L-homoserine sulfhydrylase in the art and can be called O-succinyl-L-homoserine sulfhydrylase or O-succinyl-L-homoserine thiol lyase when the additional substrate is O-succinyl-L-homoserine. Known examples of this enzyme include enzymes identified in Pseudomonas aeruginosa (MetY, Genbank accession number AAG08410; also known as PA5025 (see http://www.pseudomonas.com/ for details on nomenclature of P. aeruginosa genes); MetZ, Genbank accession number AAA83435, also known as PA3107), Thermus thermophilus (OAH1 protein, Genbank accession number B4B68505), Schizosaccharomyces pombe (Genbank accession number AAB66879) and Saccharomyces cerevisiae (MET17 protein, Genbank accession number P06106). It should be understood that other enzymes may be used in the method of the present invention, such as those known as O-acetyl-L-serine thiol lyase or O-acetyl-L-serine sulfhydrylase (also known as cysteine synthase; an example is the cysM protein of Pseudomonas aeruginosa, PA0932) which naturally use H₂S in the presence of an additional substrate such as O-acetyl-L-serine or O-succinyl-L-serine to produce cysteine. More preferably, the method of the present invention may use a stable and highly active recombinant enzyme with an appropriate affinity for H₂S.

Generally, the recycling of homocysteine or methionine by said first enzyme, homocysteine desulphurase and said second enzyme, O-acetyl-L-homoserine thiol lyase and said additional substrate O-acetyl-L-homoserine results in an overall build up and amplification of end reaction products α-ketobutyrate and ammonia and a by-product acetate. Optionally, wherein the additional substrate is O-succinyl-L-homoserine, the end reaction products are α-ketobutyrate and ammonia and the by-product is succinate.

Generally, the recycling of cysteine by said first enzyme, homocysteine desulphurase and said second enzyme, O-acetyl-L-serine thiol lyase and said additional substrate O-acetyl-L-serine results in an overall build-up and amplification of end reaction products ammonia and pyruvate and a by-product acetate. Optionally, wherein the additional substrate is O-succinyl-L-serine the end reaction products are the same however the by-product is succinate.

It should be appreciated that there may be several forms (e.g. from different organisms) or isoforms, of said first or second enzymes, for example wherein said first or second enzymes are “homocysteine desulphurase” or “O-acetyl-L-homoserine thiol lyase” respectively and all uses of such forms/isoforms are encompassed herein.

Furthermore, fragments derived from said first or second enzymes, for example as described in WO 98/07872 for homocysteine desulphurase, which display appropriate enzymatic activity, for example homocysteine desulphurase or O-acetyl-L-homoserine thiol lyase activity; or fragments derived from the nucleotide sequence encoding biologically active forms of said first or second enzymes may also be used in the method of the present invention.

Naturally, the skilled person will appreciate that such modifications mentioned hereinabove resulting in enzymatically or biologically active derivatives or fragments of said first or second enzymes are encompassed for use in the method of the present invention. Said first or second enzymes may be linked to regulatory control sequences, comprising promoters, operators, inducers, ribosome binding sites, terminators etc. and used in the production of recombinant polypeptides, for example for use in the present invention. Suitable control sequences for a given host may be selected by those of ordinary skill in the art. Additionally so-called “tagging sequences” such as additional amino acids may be added to the N or C terminus of the polypeptide, to give a so-called fusion protein upon expression of the polypeptide.

A polynucleotide fragment encoding said first or second enzyme according to the present invention may be ligated to any one or more of a variety of expression controlling DNA sequences, resulting in a so-called recombinant DNA molecule. Thus, it will be understood to the skilled man that an expression vector comprising an expressible nucleic acid molecule may be used for the transformation of a suitable host and isolation/purification of a recombinant first or second enzyme using known reagents.

In a similar manner the present invention provides the use of a hybrid comprising a first or second enzyme constructed using methods hereinbefore described. In other words, a fragment of each of said first or second enzymes may be ligated in a suitable manner in order to produce an in-frame molecule, which may be expressed, using a suitable expression vector as hereinbefore described, for example, to produce a biologically active hybrid molecule, which displays enzymatic activity of both first and second enzyme. For example, such a hybrid first/second enzyme may display both homocysteine desulphurase activity and O-acetyl-L-homoserine thiol lyase activity or homocysteine desulphurase and O-acetyl-L-serine thiol lyase activity. Advantageously, the production and use of such a hybrid first/second enzyme would allow the addition of only one molecule in order to catalyse the activities required by said first and second enzyme in the method of the present invention.

Specific vectors which can be used to clone nucleic acid sequences encoding said first or second enzymes are known in the art (e.g. Rodriguez, R. L. and D. T. Denhardt, Edit., Vectors: A Survey of Molecular Cloning Vectors and Their Uses, Butterworths, 1988).

Two specific bacterial expression vectors pQE60 and pQE30 (Qiagen Hilden, Germany) have been used for homocysteine desulphurase expression for example. The pQE series of expression vectors (e.g. pQE60 and pQE30) encode a 6 histidine tag (6xHis-tag) which enables the purification of fusion protein using metal-chelate affinity chromatography and Fast Protein Liquid Chromatography (FPLC).

The methods used in the construction of any such recombinant nucleic acid or protein molecule according to the present invention are known to the skilled addressee and are inter alia set forth in Sambrook, et al. (Molecular Cloning: A Laboratory Manual, Cold Spring Harbour Laboratory, 3^{rd} edition, 2001).

Generally, the sample may be a biological sample, e.g. of blood, plasma, faeces, saliva, vaginal fluids, urine or semen. It will be understood that homocysteine may be bound by disulphide linkage to circulating proteins, such as albumin, and homocysteine may also be present in the form of other disulphide derivatives (typically homocysteine-cysteine conjugates) in such a biological sample. To obtain an estimate of total homocysteine present in the sample it may therefore be desirable to treat the sample with a reducing agent to cleave any disulphide bonds and liberate free homocysteine. Disulphide reduction is reviewed by Jocelyn in Methods of Enzymology 143: 243-256 (1987) where a wide range of suitable reducing agents are listed. Such suitable reducing agents are incorporated in the teaching of the present invention.

In a further aspect, the method of the present invention comprises a pre-step of contacting the sample with a reducing agent. Therefore, optionally the sample of the present invention may be prior-treated with a reducing agent before using the method of the present invention. Typically, the reducing agent used is dithiothreitol (DTT), dithioerythritol (DTE) or triscarboxylethylphosphine (TCEP) to release any bound homocysteine.

The method of the present invention may also be used to estimate an analyte, which is first broken down into a metabolite, according to the present invention. For example, the concentration of the analyte is determined by measuring the amplified concentration of at least one of said reaction products produced by degradation of the metabolite by the first enzyme as produced by the cycling method of the present invention and comparing with a standard curve for metabolite levels as herein described.

Accordingly, in a further aspect, the present invention provides a cyclical method for assaying an analyte in a sample comprising the steps of:

a) adding a reducing agent to the sample to convert the analyte to a metabolite; and

b) contacting the sample with a first enzyme capable of degrading the metabolite into a plurality of reaction products; and

c) regenerating said metabolite using a second enzyme which is capable of converting at least one of said reaction product(s) obtained in b), together with an additional substrate or substrates, into said metabolite, and optionally generating an additional by-product or by-products;

d) repeating steps b) and c) for a period of time; and

e) thereafter detecting a level of a reaction product formed by enzyme degradation of said metabolite by said first enzyme, wherein the reaction product is different from the reaction product used in regenerating said metabolite, or detecting a level of said optional by-product produced by said second enzyme, or detecting a level of an additional substrate.

Typically, examples of said analytes include homocystine (where homocystine is converted to homocysteine using a reducing agent) or methionine (which may be enzymatically converted to homocysteine). In both of these cases the concentration of analyte may be determined by measurement of homocysteine.

Typically, the reducing agent used to convert homocystine to homocysteine is dithiothreitol (DTT), dithioerythritol (DTE) or triscarboxylethylphosphine (TCEP) to release covalently bound homocysteine.

In a further aspect, the methods disclosed herein may be adapted to assay for an analyte which is converted to a metabolite using the cyclic enzymatic reactions disclosed herein. Accordingly, the present invention provides a method for assaying an analyte in a sample comprising the steps of:

(a) contacting the sample with a first enzyme and one or more additional substrates, wherein the first enzyme is capable of converting the analyte into a metabolite and a reaction product;

(b) regenerating said analyte using a second enzyme which is capable of converting the metabolite, obtained in (a), into said analyte, and generating one or more additional by-products;

(c) repeating steps a) and b) for a period of time; and

(d) thereafter detecting a level of a reaction product formed by the reaction of the first enzyme and the analyte, or detecting a level of a by-product produced by said second enzyme, or detecting a level of an additional substrate.

Thus, in this aspect of the invention, a cyclic enzymatic reaction is used to amplify the level of the analyte by converting it to a metabolite such as homocysteine, cysteine or methionine. It will be apparent to the skilled person that the preferred features of other aspects of the invention disclosed herein are applicable to this method, for example with the first enzyme disclosed in relation to other aspects of the invention corresponding to the second enzyme in this aspect of the invention, and vice versa.

In this aspect of the invention, preferably the analyte is hydrogen sulphide. The first enzyme is preferably O-acetyl-L-homoserine thiol lyase or O-acteyl-L-serine thiol lyase and the second enzyme is a homocysteine desuphurase. preferred additional substrates include O-acetyl-L-homoserine and O-succinyl L-homoserine.

The reaction product may be acetate or succinate and the one or more additional by-products may be α-ketobutyrate or ammonia.

Conveniently, any of the reaction products of the method of the present invention described hereinbefore for example, α-ketobutyrate, hydrogen sulphide, methanethiol and/or ammonia, may be determined using any of a variety of suitable methods known to the skilled person. Typically, the amplified products and not those reaction products used in the recycling of metabolite may be detected. Methods suitable for such detection include, for example, calorimetric, spectrophotometric, electrochemical, fluorimetric or luminescent methods. Preferably, the method is sensitive enough to detect concentration of <5 μmol/l homocysteine in a sample.

Alternatively, said additional substrates of the method of the present invention may be labelled in some way in order to detect any of the end reaction products or by-products of the present invention as hereinbefore described. Labelling may be by fluorogenic label or isotopic label, in particular C¹³.

The optional generation of additional by-product or by-products according to the present invention include any such by-products produced in the regeneration of metabolite or the catalyses by a second enzyme and addition of an additional substrate. For example, wherein the metabolite is homocysteine or methionine, said second enzyme is O-acetyl-L-homoserine thiol lyase and the additional substrate is O-acetyl-L-homoserine then the by-product is acetate. For the same metabolite and second enzyme, but where the additional substrate is O-succinyl-L-homoserine, the by-product is succinate.

Wherein the metabolite is cysteine, said second enzyme is O-acetyl-L-serine thiol lyase and said substrate is O-acetyl-L-serine the by-product produced is acetate. For the same metabolite and second enzyme but wherein the substrate used is O-succinyl-L-serine the by-product produced is succinate.

Advantageously, the by-products of the method of the present invention as hereinbefore described may be detected using any of a variety of suitable methods known to the skilled person. For example, acetate may be detected using the enzyme acetate thio-kinase whereas succinate may be detected using fumarate reductase or succinate dehydrogenase.

α-ketobutyrate generated by the degradation of homocysteine may be detected following the method of Soda (Soda, K. (1968) Anal. Biochem. 25: 228-235) using 3-methyl-2-benzothiazolone hydrazone hydrochloride (MBTH).

An additional method of determining α-ketobutyrate is described by Li, R.+Kenyon, G. L. (1995). A spectrophotometric determination of α-dicarboxyl compounds and its application to the enzymatic formation of α-ketobutyrate. Analytical Biochemistry 230 37-40.

A particularly preferred method of detecting α-keto-butyrate is by adding NADH and lactate dehydrogenase so as to convert the α-ketobutyrate to α-hydroxybutyrate with the generation of NAD⁺. The level of NAD⁺ can then be measured by a number of methods involving conversion to NADH including spectrophotometrically by absorbance at 340 nm; fluorescently by excitation at 365 nm and emission at 460 nm (Palmer T. (1991) Understanding Enzymes 3rd Edition, Ellis Horwood, London); calorimetrically using tetrazolium salts (Altman, P. F. (1974) Histochemistry 38 p 155-171); electrochemically (Morroux J. Elring P J (1979) Anal Chem 51, 346; Blaedel W J, Jenkins R A (1975) Anal Chem 47, 1335; Juegfeldt H et al (1981) Anal Chem 53, 1979; Wang J, Lin M S (1987) Electroanal Chem 221, 257); and luminescently (Whitehead T P et al (1979) Clin Chem 25, 1531).

Hydrogen sulphide generated by homocysteine degradation may be determined, for example, by reacting with lead acetate to produce lead sulphide according to the following (stoichiometric) equation.
H₂S+Pb(CH₂COOH)₂→PbS+2CH₃COOH

Lead sulphide produced may then be measured spectrophotometrically at a suitable wavelength, such as A360 nm. (Thong K. W+Coombs, G. H. (1985) Homocysteine desulphurase activity in trichomonads. IRCS Medical Science 13: 493-494).

Alternatively hydrogen sulphide, may be measured using the methylene blue method as described by Clime, J. D. Limnol, Oceanogr. (1969) 14: 454-458. Briefly, hydrogen sulphide is reacted with 0.17 mM N, N-dimethyl-p-phenylene diamine sulphate in acid and ferric chloride in acid to produce methylene blue which can be detected spectrophotometrically at 650-670 nm.

Ammonia generated by the degradation of homocysteine may be reacted with phenol in the presence of hypochlorite to produce indophenol as described by Horn, D. B.+Squire, C. R. (1967), An improved method for the detection of ammonia in blood plasma Clin. Chem. Acta 17 99-105. Indophenol so produced may then be detected spectrophotometrically at a suitable wavelength, for example, 570 nm.
NH₃+OCl+phenol→indophenol

Further, methods for detecting ammonia include: enzymatically, using α-ketoglutarate and NAD(P)H with glutamate dehydrogenase as described by Mondzac A et al (1965) J. Lab. Clin. Med. 66 526; electrochemically using an ammonia electrode as described by Guilbault et al (1985) Anal. Chem. 57 2110; using 2-oxoglutarate and NADH to generate glutamate, water and NAD⁺ and then measuring NAD⁺ as described above; and adding silver nitrate to ammonia to generate a black precipitate.

The by-product acetate generated by the recycling of metabolite by said second enzyme and degradation of metabolite by said first enzyme as hereinbefore described may be detected by using HPLC and enzyme based assays known to the skilled person (King, G. M., 1991), including the use of acetate thiokinase.

Similarly, the by-product succinate generated by the recycling of metabolite by said second enzyme and degradation of metabolite by said first enzyme as hereinbefore described may be detected by using enzyme based assays known to the skilled person, including the use of the enzyme succinate dehydrogenase.

It should be understood that homocysteine desulphurase may also be used to assay for enzymes that catalyse reactions involving metabolite, as hereinbefore described, as either substrate or product (for instance S-adenosylhomocysteine hydrolase). For example, the metabolite homocysteine may be assayed in the ways applied to its detection in the biological samples as hereinbefore described. Similarly homocysteine desulphurase may be used to assay enzymes that catalyse reactions involving methionine or cysteine or related compounds as substrates or products. These metabolites could be assayed via their conversion to α-keto acids by homocysteine desulphurase and the measurement of the α-keto acids as described previously.

In a further aspect, the present invention provides a kit for diagnostic in vitro determination of a metabolite in a sample, wherein the kit comprises a first enzyme capable of degrading the metabolite into a plurality of reaction products and a second enzyme which is capable of converting at least one of said reaction product(s) obtained, together with an additional substrate or substrates, into said metabolite, and optionally generating an additional by-product or by-products.

Preferably, said kit according to the present invention further comprises means for enabling detection of any of the reaction product(s) as hereinbefore described.

Preferably, the first enzyme is homocysteine desulphurase as hereinbefore described.

Conveniently, the kit may provide for in vitro determination of homocysteine, methionine or cysteine as hereinbefore described, wherein the first enzyme in the kit is homocysteine desulphurase.

Preferably, the second enzyme is an enzyme capable of resynthesising any of said reaction product(s) to the metabolite, homocysteine, methionine or cysteine. More preferably, the second enzyme is an enzyme as hereinbefore described, which utilises an additional substrate and produces-a by-product as hereinbefore described. Typically, the reaction product to be recycled is hydrogen sulphide.

Optionally, said kit may further comprise a reducing agent, as hereinbefore described, for converting an analyte in a sample to said metabolite. It will be understood therefore, as previously described that the presence of said reducing agent may allow the detection of said analyte through conversion to metabolite and detection of said reaction product(s).

Other modifications designed to improve the aforementioned methods and/or kit may also be employed, which may serve to reduce any background signal and/or improve stability of reagents employed in the assay. For example, it may be desirable to bind, oxidise and/or depotentiate the reducing agent after the reducing agent has served its purpose. It may also be appropriate to utilise a cryo/lyoprotectant and/or a proteinaceous or non-proteinaceous stabilising agent. Additionally, it is possible to use an agent, which serves to deactivate pyruvate or undesirable keto acids. Moreover, it may be convenient to bind any of the reagents to a substrate prior to conducting any assay or diagnostic in vitro detection using the kit of the present invention. Suitable methods to achieve any/all of the above are described in WO 01/77670, to which the skilled person is directed.

Advantageously, the method of the present invention may be used to assay for a metabolite using a cycling assay as hereinbefore described wherein said reaction product(s), which is/are different to the reaction product used to regenerate said metabolite, or said optional by-product is/are amplified and therefore due to their amplified concentration in said sample can be more easily and accurately detected and quantified as compared to reaction products generated for detection and quantification of a metabolite in a known ‘non-cycling’ method.

Embodiments of the present invention will now be described in more detail by way of examples and not limitation with reference to the accompanying figure and examples.

### BRIEF DESCRIPTION OF FIGURES

FIG. 1 shows a schematic diagram of the reactions involved in the method of the present invention, wherein for this particular embodiment, the first enzyme is homocysteine desulphurase, the second enzyme is O-acetyl-L-homoserine thiol lyase and the additional substrate is O-acetyl-L-homoserine.

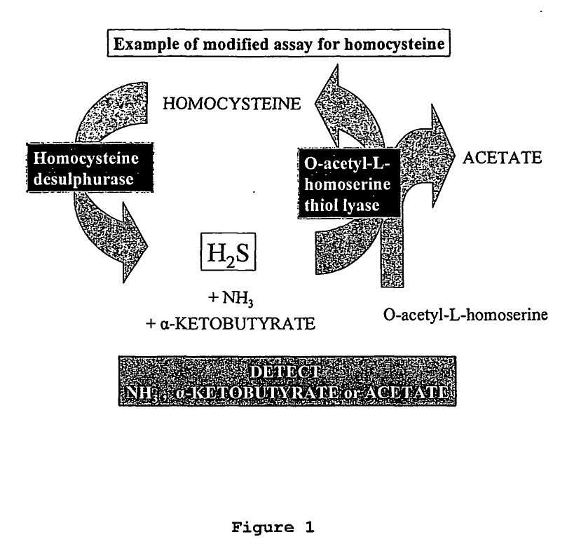

### DETAILED DESCRIPTION

Cyclical Methods to Assay the Metabolite Homocysteine

Assay I

Assay Principle

Homocysteine levels can be measured using recombinant homocysteine desulphurase prepared according to the previous examples in 66 mM sodium phosphate buffer pH 7.5 by measuring the ammonia produced. Dithiothreitol (DTT) is initially used to break homocystine down into homocysteine and to release protein bound homocysteine or homocystine. Homocysteine desulphurase catalyses the conversation of homocysteine to α-ketobutyrate, ammonia and hydrogen sulphide. The hydrogen sulphide is converted back to homocysteine by the action of O-acetyl-L-homoserine thiol lyase. The resulting homocysteine is converted to α-ketobutyrate, ammonia and hydrogen sulphide. Thus, the amount of ammonia is increased progressively. This is subsequently quantified by the addition of phenol and hypochlorite (Horn and Squire, 1967).

Assay reagents

| | | |
|---|---|---|
| 0.1 M sodium phosphate | 0.5 | ml |
| homocysteine 5 μM to 50 μM | 0.49 | ml |
| (final concentration) | | |
| homocysteine desulphurase (20000U/l) | 10 | μl |
| O-acetyl L-homoserine thiol lyase (50000U/l) | 10 | μl |
| O-acetyl-L-homoserine (500 mM) | 10 | μl |

The mixture was incubated at 37° C. for 10 minutes before the addition of phenol and hypochlorite to allow the colour to develop. The homocysteine levels were then determined by measuring the absorbance at 570 nm.

Results

| Homocysteine concentrations | |
|---|---|
| (μM) | Absorbance |
| 0 | 0.08 |
| 5 | 0.21 |
| 10 | 0.36 |
| 15 | 0.47 |
| 20 | 0.61 |
| 30 | 0.86 |

Assay II
Assay Principle

Dithiothreitol (DTT) is initially used to break homocystine down into homocysteine and to release protein bound homocystine and homocysteine. The homocysteine is then degraded into α-ketobutyrate, NH₃ and H₂S by the action of homocysteine desulphurase. The hydrogen sulphide is converted back to homocysteine by reaction of O-acetyl L-homoserine thiol lyase. A specific lactate dehydrogenase isoenzyme is then utilised to convert α-ketobutyrate into α-hydroxybutyrate with the concomitant release of NAD⁺. After removal of any NADH by lowering of the pH using HCl the NAD⁺ is fed into a cycling mechanism involving ethanol, alcohol dehydrogenase, diaphorase and tetrazolium salts to generate a coloured product which can be photometrically measured. The increase in colour corresponds to the concentration of homocysteine in the sample.

Performance of the Assay

Step 1: Mix 20 μl of sample (e.g. citrated plasma) with 500 μl 0.1 mol/l HEPES, 1.0 mmol/l NADH, 20,000 μmoles/min/l homocysteine desulphurase, 50,000 U/l O-acetyl-L-homoserine thiol lyase, 5 mmol/l O-acetyl-L-homoserine, 50,000 U/l lactate dehydrogenase and 0.05 mol/l dithiothreitol, pH 8.0 into a cuvette. Incubate at 37° C. for 3 min.

Step 2: Add 500 μl 1 mol/l HCl, 0.55% (v/v) Nonidet P40, 1×10⁻⁴ mol/l nitroblue tetrazolium (NBT). Incubate at 37° C. for 5 min.

Step 3: Add 500 μl Tris(hydroxymethylaminomethane) (TRIS), 1 mol/l ethanol followed by 50 μl 20,000 U/l alcohol dehydrogenase, 1000 U/l diaphorase. Measure the increase in absorbance at 527 nm for 5 minutes after the addition of the reagent containing alcohol dehydrogenase.

Assay Performance

i) Standard Curve

A typical standard curve is shown in the table below:

| Homocysteine concentration | |
|---|---|
| (μmol/l) | Absorbance |
| 0 | 0.161 |
| 5 | 0.276 |
| 10 | 0.391 |
| 15 | 0.504 |
| 20 | 0.611 |
| 25 | 0.731 |

Standard curve was generated by spiking homocysteine into serum. The background signal is in part caused by endogenous levels of homocysteine

ii) Sensitivity

It is clearly possible to detect concentrations of <5 μmol/l homocysteine.

Assay III

Assay Principle

Homocysteine desulphurase catalyses the conversation of homocysteine to α-ketobutyrate, ammonia and hydrogen sulphide. The hydrogen sulphide is converted back to homocysteine-by the action of O-acetyl-L-homoserine thiol lyase. The resulting homocysteine is converted to α-ketobutyrate, ammonia and hydrogen sulphide. Thus, the amount of ammonia is increased progressively. This is quantified by the addition of ketoglutarate, NADH and glutamate dehydrogenase and monitoring the oxidation of NADH.

Contents (in 1 ml): 50 mM sodium phosphate, pH 7.8; 5 mM ketoglutarate; 100 μM NADH; 230 μg glutamate dehydrogenase (Roche Diagnostics); O-succinyl-L-homoserine 0.3 mM; homocysteine sample; 6 μg O-acetyl-L-homoserine thiol lyase; 2.6 μg homocysteine desulphurase (MGL2).

Procedure: 10 min preincubation at 37° C. before addition of homocysteine desulphurase to start reaction. Incubation at 37° C., monitor absorbance at 340 nm. 30 min incubation, measure rate over 10-30 min period.

Results

| | | Absorbance change |
|---|---|---|
| Homocysteine | Absorbance change | over 20 minutes |
| (micromolar) | over 20 minutes | greater than control |
| 0 | 0.135 | Not applicable |
| 5 | 0.153 | 0.018 |
| 10 | 0.173 | 0.038 |
| 25 | 0.232 | 0.097 |

Features of this Assay

The assay uses a single reaction for determination of homocysteine concentration, with pre-incubation to release bound homocysteine. The measurements made in the assay were kinetic. The assay was capable of detection 2.5 micromolar homocysteine, and potentially lower concentrations.

O-Acetyl-L-Homoserine Thiol Lyase Enzymes Used

Two enzymes from Pseudomonas aeruginosa have been used, those encoded by genes with the numbers PA5025 and PA3107. Production of recombinant proteins encoded by PA5025 and PA3107 used the following reagents and conditions.
PET28a+ (N-terminal His-Tag)
Host strain: BL21/DE3.
Grow at 37° C. to A₆₀₀ 0.6-0.8.
Induce expression with 2 mM IPTG
Harvest cells after 4 h at 37° C.
Sonicate in 50 mM NaH₂PO₄, 300 mM NaCl, 20 micromolar pyridoxal phosphate
Purify His-tagged protein by nickel agarose chromatography
Elute with a gradient of 75-500 mM imidazole.

The peak fractions were combined and dialysed two times against: 50 mM potassium phosphate, 1 mM EDTA, 0.2 mM pyridoxal phosphate, pH 7.8 (100× volume for 90 min at 4° C.). Add sodium azide to 0.02w and store at 4° C.

Properties of recombinant proteins

| | Concentration | Yield | Stability at |
|---|---|---|---|
| Protein | (mg/ml) | (mg/l) | 4° C. (weeks) |
| PA5025 | 1.2 | 6 | >6 |
| PA3107 | 2.4 | 14 | >6 |

Activities of Recombinant Enzymes

Specific activities (micromol/min/mg protein) towards different substrates

| | O-acetyl- | O-acetyl-L- | O-succinyl-L- |
|---|---|---|---|
| Protein | serine | homoserine | homoserine |
| PA5025 | <0.1 | 44 | 60 |
| PA3107 | <0.1 | <0.1 | 139 |

Assayed according to Kredich and Tomkins (Journal of Biological Chemistry 241, 4955-4965, 1966)

Kinetic properties of recombinant enzymes

| | | O-acetyl-L- | O-succinyl-L- | |
|---|---|---|---|---|
| | | homoserine | homoserine | Na₂S |
| PA5025 | Vₘₐₓ | 42.1 ± 6.1 (4) | 70.4 ± 9.3 (3) | 73.2 ± 6.1 (3) |
| | Kₘ | 1.8 ± 0.5 (4) | 1.1 ± 0.1 (3) | 0.2 ± 0.1 (3) |
| PA3107 | Vₘₐₓ | | 180.4 ± 21.0 (3) | 174.2 ± 39.8 (3) |
| | Kₘ | | 4.4 ± 1.0 (3) | 0.8 ± 0.2 (3) |

Vₘₐₓ (μmol/min/mg protein ± SD); Kₘ (mM ± SD); n in brackets.
Kₘ for O-acetyl-L-homoserine was determined with 3 mM Na₂S.
Kₘ for Na₂S was determined with 15 mM O-succinyl-L-homoserine.

### REFERENCES

The documents referred to herein are all expressly incorporated by reference in their entirety.

1. Ueland, P. M. (1992) Plasma homocysteine and cardiovascular disease. In: Athersclerotic cardiovascular disease (Francis, R. N. ed.), pp. 183-230. Marcel Decker. New York.

2. Kluijtmans, L. A. J, et al. (1996) Molecular genetic analysis of mild hyperhomocysteinemia: A common mutation in the methylenetetrahydrofolate reductase gene is a genetic risk factor for cardiovascular disease. AM. J. Hum. Genet.58, 35.

3. Cravo, M. L. et al. (1996) Hyperhomocysteinemia in chronic alcoholism: correlation with folate, vitamin B-12 and vitamin B-6 status. Am. J. Clin. Nutr. 63,220.

4. Bostom, A. G. et al (1996) High dose B-vitamin treatment of hyperhomocysteinemia in dialysis patients, Kidney International 49, 147.

5. Steegers-Theunissen, R. P. M. (1992) Hyperhomocysteinaemia and recurrent spontaneous abortion or abruptioplacentae. Lancent 339,1122.

6. Mudd, S. H. (1989) Disorders of transsulphuration. In: The metabolic basis of inherited disease (Scriver, C. R. ed.) pp. 693-734. McGraw-Hill. New York.

7. te Poele-Pothoff, M. T. et al (1995) Three different methods for the determination of total homocysteine in plasma. Ann. Clin. Biochem. 32, 218.

8. Hori, H., Takabayashi, K., Orvis, L., Carson, D. A., Nobori, T. (1996) Gene cloning and characterisation of Pseudomonas putida L-methionine-alpha-deamino-gamma-mercaptomethane-lyase. Cancer Research 56 No. 9 pp 2116-2122.

9. Sambrook, J., Fritsch, E. F. and Maniatis, T. (1989) Molecular Cloning, A laboratory manual (2nd ed.). Cold Spring Harbor Laboratory Press.

10. Ono, B. I., Tanaka, K., Naito, K., Heike, C., Shinoda, S., Yamamoto, S., Ohmori, S., Oshima, T. and Toh-E, A. (1992) Cloning and characterization of the CYS3 gene of Saccharaomyces cerevisiae. Journal of Bacteriology 174, 3339-3347.

11. Lu, Y., O'Dowd, B. F., Orrego, H. and Israel, Y. (1992) Cloning and nucleotide sequence of human liver cDNA encoding for cystathionine gamma-lyase. Biochemical and Biophysical Research Communications 189, 749-758.

12. Lockwood, B. C. and Coombs, G. H (1991) Purification and characterisation of methionine gamma-lyase from Trichomonas vaginalis. Biochemical Journal. 279, 675-682.

13. Erickson. P. F., Maxwell, I. H., Su, L. J., Baumann, M. and Glode, L. M. (1990) Sequence of cDNA for cystathionine gamma-lyase and comparison of deduced amino acid sequence with related Escherichia coli enzymes. Biochemical Journal 269, 335-340.

14. Lockwood, B. C., North, M. J. and Coombs, G. H. (1984) Trichomonas vaginalis, Tritrichomonas foetus and Trichomitus Batrachorum: Comparative proteolytic activity. Experimental Parasitology 58, 245-253.

15. Shipchandler, M. T., and Moore E. G. (1995). Rapid, fully automated measurement of plasme homocyst(e)ine with Abbott Imx analyser. Clin Chem. 41 (7), 991-4

16. Frantzen, F., Farren, A. L., Alfheim, I., and Nordhei, A. K., 1998. Enzyme conversion immunoassay for determining total homocysteine in plasma or serum. Clin Chem., 44 (2), 311-6

17. Brzywczy, J., Sienko, A., Kucharska, A., and Paszewski, A., 2002. Sulphur amino acid synthesis in Schizosaccharomyces pombe represents a specific variant of sulphur metabolism in fungi. Yeast 19, 29-35

18. Foglino, M., Borne, F., Bally, M., Ball, G., and Patte, J. C., 1995. Ad irect sulfhydrylation pathway is used for methionine biosynthesis in Pseudomonas aeruginosa. Microbiology, 141, 431-439

19. Shimizu, H., Yamagata, R., Masui, Y., Inoue, T., Shibata, S., Yokoyama, S., Kuramitsu, S., and Iwama, T., 2001. Cloning and overexpression of the oah1 gene encoding O-acetyl-L-homoserine sulfhydrylase of Thermus thermophilus HB8 and characterisation of the gene product. Biochim. Biophys. Acta 1549, 61-72

20. King, G. M., 1991. Measurement of acetate concentrations in marine pore water by using an enzymatice approach. Appl. Environ. Microbiol., 57, 3476-3481

1. A method for assaying a metabolite in a sample comprising the steps of:
a) contacting the sample with a first enzyme capable of degrading a metabolite into a plurality of reaction products;
b) regenerating said metabolite using a second enzyme which is capable of converting at least one of said reaction product(s) obtained in a), together with an additional substrate or substrates, into said metabolite, and optionally generating an additional by-product or by-products;
c) repeating steps a) and b) for a period of time; and
d) thereafter detecting a level of a reaction product formed by enzyme degradation of said metabolite by said first enzyme, wherein the reaction product is different from the reaction product used in regenerating said metabolite, or detecting a level of said optional by-product produced by said second enzyme or detecting a level of an additional substrate.

2. A cyclical method for assaying an analyte in a sample comprising the steps of:
a) adding a reducing agent to the sample to convert an analyte to a metabolite;
b) contacting the sample with a first enzyme capable of degrading the metabolite into a plurality of reaction products; and
c) regenerating said metabolite using a second enzyme which is capable of converting at least one of said reaction product(s) obtained in b), together with an additional substrate or substrates, into said metabolite, and optionally generating an additional by-product or by-products;
d) repeating steps b) and c) for a period of time; and
e) thereafter detecting a level of a reaction product formed by enzyme degradation of said metabolite by said first enzyme, wherein the reaction product is different from the reaction product used in regenerating said metabolite, or detecting a level of said optional by-product produced by said second enzyme, or detecting a level of an additional substrate.

3. The method of claim 1, wherein the metabolite is homocysteine, methionine or cysteine.

4. The method of claim 2, wherein the analyte is homocystine and the metabolite is homocysteine.

5. The method of claim 3, wherein the additional substrate is (i) O-acetyl-L-homoserine or O-succinyl-L-homoserine where the metabolite is homocysteine or methionine or (ii) O-acetyl-L-serine or O-succinyl-L-serine where the metabolite is cysteine.

6. The method of claim 1, wherein amplification of the metabolite in the assay is 2- to 1000-fold.

7. The method of claim 3, wherein the first enzyme is homocysteine desulphurase.

8. The method of claim 7, wherein:
(i) when the metabolite is homocysteine, the reaction products are α-ketobutyrate, ammonia (NH₃) and hydrogen sulphide (H₂S); or (ii) when the metabolite is methionine, the reaction products are α-ketobutyrate, ammonia and methanethiol; or
(iii) when the metabolite is cysteine, the reaction products are pyruvate, ammonia, and hydrogen sulphide.

9. The method of claim 3, wherein the second enzyme utilises hydrogen sulphide in the synthesis of cysteine or homocysteine or methanethiol in the synthesis of methionine.

10. The method of claim 9, wherein:
(i) the second enzyme is O-acetyl-L-homoserine thiol lyase which is capable of using hydrogen sulphide to synthesise homocysteine and said additional substrate is O-acetyl-L-homoserine or O-succinyl-L-homoserine; or
(ii) the second enzyme is O-acetyl-L-homoserine thiol lyase which is capable of using methanethiol to synthesise methionine and said additional substrate is O-acetyl-L-homoserine or O-succinyl-L-homoserine; or
(iii) the second enzyme is O-acetyl-L-serine thiol lyase which is capable of using hydrogen sulphide to produce cysteine and said additional substrate is O-acetyl-L-serine or O-succinyl-L-serine.

11. The method of claim 3, wherein the metabolite is homocysteine or methionine, the first enzyme is homocysteine desulphurase, the second enzyme is O-acetyl-L-homoserine thiol lyase, the additional substrate O-acetyl-L-homoserine, the reaction products are α-ketobutyrate and ammonia and the by-product is acetate.

12. The method of claim 3, wherein the metabolite is homocysteine or methionine, the first enzyme is homocysteine desulphurase, the second enzyme is O-acetyl-L-homoserine thiol lyase, the additional substrate is O-succinyl-L-homoserine, the reaction products are α-ketobutyrate and ammonia and the by-product is succinate.

13. The method of claim 3, wherein the metabolite is cysteine, the first enzyme is homocysteine desulphurase, the second enzyme is O-acetyl-L-serine thiol lyase, the additional substrate is O-acetyl-L-serine, the reaction products are ammonia and pyruvate and the by-product is acetate.

14. The method of claim 3, wherein the metabolite is cysteine, the first enzyme is homocysteine desulphurase, the second enzyme is O-acetyl-L-serine thiol lyase, the additional substrate is O-succinyl-L-serine, the reaction products are ammonia and pyruvate and the by-product is succinate.

15. The method of claim 1, wherein the method comprises a pre-step of contacting the sample with a reducing agent to release bound metabolite.

16. The method of claim 15, wherein the reducing agent is dithiothreitol (DTT), dithioerythritol (DTE) or triscarboxylethylphosphine (TCEP).

17. The method of claim 3, wherein the method is capable of detecting a concentration of <5 μmol/l homocysteine in a sample.

18. A method for assaying an analyte in a sample comprising the steps of:
a) contacting the sample with a first enzyme and one or more additional substrates, wherein the first enzyme is capable of converting the analyte into a metabolite and a reaction product;
b) regenerating said analyte using a second enzyme which is capable of converting the metabolite, obtained in (a), into said analyte, and generating one or more additional by-products;
c) repeating steps a) and b) for a period of time; and
d) thereafter detecting a level of a reaction product formed by the reaction of the first enzyme and the analyte, or detecting a level of a by-product produced by said second enzyme, or detecting a level of an additional substrate.

19. The method of claim 18, wherein the metabolite is homocysteine, cysteine or methionine.

20. The method of claim 18, wherein the analyte is hydrogen sulphide.

21. The method of claim 18, wherein:
(i) the first enzyme is O-acetyl-L-homoserine thiol lyase or O-acteyl-L-serine thiol lyase and the second enzyme is a homocysteine desuphurase; and/or
(ii) the additional substrates is O-acetyl-L-homoserine or O-succinyl L-homoserine; and/or
(iii) the reaction product is acetate or succinate; and/or
(iv) the one or more additional by-products is α-ketobutyrate or ammonia.

22. The method of claim 18, wherein the first and/or second enzymes are enzymatically active protein fragments.

23. The method of claim 18, wherein the sample is a biological sample of blood, plasma, faeces, saliva, vaginal fluids, urine or semen.

24. The method of claim 18, wherein the additional substrate is labelled.

25. The method of claim 25, wherein the label is a fluorogenic label or an isotopic label.

26. The method of claim 18, wherein the level of the reaction product, by-product or additional substrate is detected using a colorimetric, spectrophotometric, electrochemical, fluorimetric or luminescent method.

27. The method of claim 18, wherein the step of detecting the reaction product or the by-product comprises:
(i) detecting acetate using HPLC or the enzyme acetate thio-kinase; or
(ii) detecting succinate using the enzymes fumarate reductase or succinate dehydrogenase; or
(iii) detecting α-ketobutyrate using 3-methyl-2-benzothiazolone hydrazone hydrochloride (MBTH); or
(iv) detecting α-keto-butyrate by adding NADH and lactate dehydrogenase to convert the α-ketobutyrate to α-hydroxybutyrate with the generation of NAD⁺ and measuring the level of NAD⁺; or
(v) detecting hydrogen sulphide by reacting with lead acetate to produce lead sulphide and measuring the lead sulphide produced spectrophotometrically; or
(vi) detecting hydrogen sulphide using the methylene blue method; or
(vii) detecting ammonia by reacting with phenol in the presence of hypochlorite to produce indophenol and measuring the indophenol spectrophotometrically; or
(ix) detecting ammonia using the enzyme α-ketoglutarate and NAD(P)H with glutamate dehydrogenase, electrochemically using an ammonia electrode, using 2-oxoglutarate and NADH to generate glutamate, water and NAD⁺ and then measuring NAD⁺, or adding silver nitrate to ammonia to generate a black precipitate.

28. A kit for diagnostic in vitro determination of a metabolite in a sample, wherein the kit comprises a first enzyme capable of degrading the metabolite into a plurality of reaction products and a second enzyme which is capable of converting at least one of said reaction product(s) obtained, together with an additional substrate or substrates, into said metabolite, and optionally generating an additional by-product or by-products.

29. The kit of claim 28, wherein the metabolite is homocysteine, methionine or cysteine.

30. The kit of claim 28, wherein the first enzyme is homocysteine desulphurase.

31. The kit of claim 28, wherein the second enzyme is O-acetyl-L-homoserine thiol lyase or O-acetyl-L-serine thiol lyase.

32. The kit of claim 28, wherein the additional substrate is O-acetyl-L-homoserine or O-succinyl-L-homoserine where the metabolite is homocysteine or methionine, or O-acetyl-L-serine or O-succinyl-L-serine where the metabolite is cysteine.

33. The kit of claim 28, further comprising detection means for detecting a reaction product, a by-product or an additional substrate.

34. The kit of claim 28, further comprising a reducing agent for converting an analyte in a sample to said metabolite.
